(19) United States
(12) Patent Application Publication
(10) Pub. No.: US 2023/0303762 A1
(43) Pub. Date: Sep. 28, 2023

(54) LIGHT-EMITTING MARKER

(71) Applicant: Sumitomo Chemical Co., Ltd., Tokyo (JP)

(72) Inventors: Kiran Kamtekar, Godmanchester (GB); Nazrul Islam, Godmanchester (GB); Sheena Zuberi, Godmanchester (GB); Jonathan Behrendt, Godmanchester (GB)

(73) Assignee: Sumitomo Chemical Co., Ltd., Tokyo (JP)

(21) Appl. No.: 18/020,053

(22) PCT Filed: Aug. 6, 2021
(86) PCT No.: PCT/EP2021/072092

§ 371 Date: Feb. 6, 2023

(30) Foreign Application Priority Data
Aug. 7, 2020 (GB) 2012310.5

(51) Int. Cl. C08G 61/12; C09K 11/06; G01N 33/58; G01N 21/64

(52) U.S. Cl. CPC C08G 61/124; C09K 11/06; G01N 33/582; G01N 21/6428; C09K 2211/1491; G01N 2021/6439

(57) ABSTRACT

A conjugated light-emitting polymer comprising a host repeat unit and an intermediate repeat unit in a backbone of the conjugated light emitting polymer, and an emissive unit. The host repeat unit has a wider band gap than the intermediate unit. The intermediate repeat unit has a wider band gap than the emissive unit. The emissive unit is either: an emissive repeat unit of a block copolymer wherein the block copolymer comprises a first block comprising the intermediate repeat unit and the emissive repeat unit and a second block comprising the host repeat unit; a substituent of a proportion of the host repeat units and at least some of the host repeat units substituted with an emissive unit are arranged directly adjacent to an intermediate repeat unit; a substituent of a proportion of the intermediate repeat units; or a repeat unit in the backbone of the conjugated polymer and is arranged directly adjacent to the intermediate repeat unit. The conjugated light-emitting polymer may be part of a light-emitting marker for detecting the presence of a target analyte in a sample.

Embodiments of the present disclosure relate to light-emitting polymers and use thereof in detection of a target analyte.

Light-emitting polymers for marking a target analyte are known.

Fischer et al, “Enhanced Brightness Emission-Tuned Nanoparticles from Heterodifunctional Polyfluorene Building Blocks”, J. Am. Chem. Soc. 2013, 135, 3, 1148-1154 discloses nanoparticles of perylene-end-capped polyfluorene block copolymers containing a terrylene diimide dye for an energy cascade resulting in emission exclusively in the deep red and near-infrared regime.

U.S. Pat. No. 5,763,189 discloses particles comprising an energy donor as a first component and a fluorescent dye as a second component positioned in said particles at an energy exchanging distance from one another, wherein the two components have a Stokes shift of greater than or equal to 50 nm.

U.S. Pat. No. 5,573,909 discloses microparticles having a series of two or more fluorescent dyes having overlapping excitation and emission spectra.

Zhang et al “High-intensity near-IR fluorescence in semiconducting polymer dots achieved by cascade FRET strategy”, Chem Sci. 2013 May 1; 4(5):2143-2151 discloses multi-component semiconducting polymer dots.

U.S. Pat. No. 6,545,164 discloses low molecular weight fluorescent labelling complexes with large wavelength shifts between absorption of one dye in the complex and emission from another dye in the complex.

WO 2020/058440 discloses a particle comprising an inorganic matrix material; a first light-emitting material; and a second light-emitting material, wherein the first light-emitting material is a light-emitting polymer. The first light-emitting material may transfer excitation energy to the second light-emitting material.

WO 2020/058123 discloses a particle comprising an inorganic matrix material and a light-emitting polymer having a light-emitting group and a host repeat unit.

### SUMMARY

In some embodiments, the present disclosure provides a conjugated light-emitting polymer comprising a host repeat unit and an intermediate repeat unit in a backbone of the conjugated light emitting polymer and an emissive unit wherein:
the host repeat unit has a wider band gap than the intermediate unit;
the intermediate repeat unit has a wider band gap than the emissive unit; and
the emissive unit is either:
   an emissive repeat unit of a block copolymer wherein the block copolymer comprises a first block comprising the intermediate repeat unit and the emissive repeat unit and a second block comprising the host repeat unit;
   a substituent of a proportion of the host repeat units and at least some of the host repeat units substituted with an emissive unit are arranged directly adjacent to an intermediate repeat;
   a substituent of a proportion of the intermediate repeat units; or
   a repeat unit in the backbone of the conjugated polymer and is arranged directly adjacent to the intermediate repeat unit.

Optionally, the emissive unit is a substituent of a proportion of the intermediate repeat units.

Optionally, the emissive unit is a substituent of a proportion of the host repeat units and at least some of the host repeat units substituted with an emissive unit are arranged directly adjacent to an intermediate repeat.

Optionally, the emissive unit is a repeat unit in the backbone of the polymer. Optionally, the the polymer is the block copolymer. Optionally, the first block further comprises some of the host repeat units. Optionally, the second block consists of the host repeat units.

Optionally, the host repeat unit is selected from a repeat unit of formula (I); a repeat unit of formula (II); and an arylene repeat unit which is unsubstituted or substituted with one or more substituents:

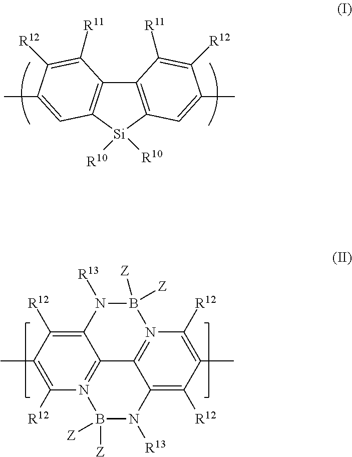

wherein R¹⁰ in each occurrence is independently a substituent; R¹¹ in each occurrence is independently H or a substituent and two R¹¹ groups may be linked to form a ring; R¹² independently in each occurrence is H or a substituent; R¹³ independently in each occurrence is a C₁₋₂₀ hydrocarbyl group; and Z in each occurrence is independently a substituent.

Optionally, the host repeat unit is an arylene repeat unit selected from repeat units of formulae (III)-(VI):

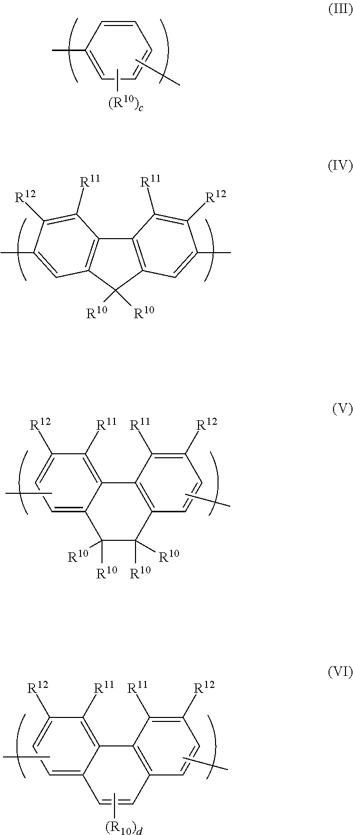

wherein c is 0, 1, 2, 3 or 4; and d is 0, 1 or 2.

Optionally, the intermediate repeat unit comprises one or more heteroarylene units in the polymer backbone wherein each of the one or more heteroarylene units is independently unsubstituted or substituted with one or more substituents.

Optionally, the intermediate repeat unit is selected from repeat units of formulae (IX)-(XII):

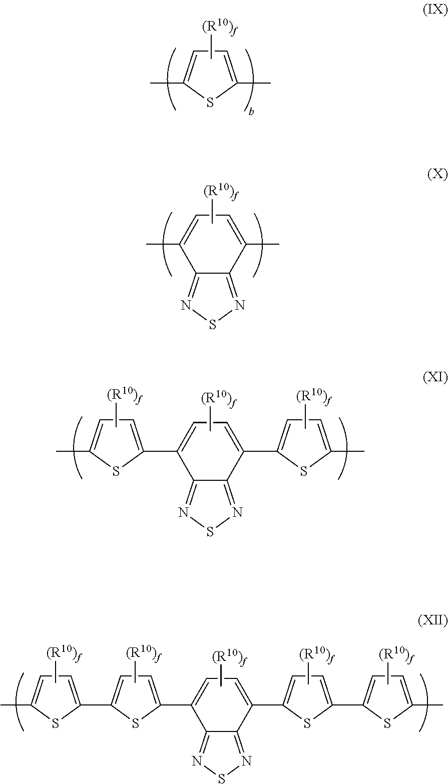

wherein R¹⁰ in each occurrence is independently a substituent, and f in each occurrence is independently 0, 1 or 2.

Optionally, the emissive unit has formula (XXX) or (XXXI):

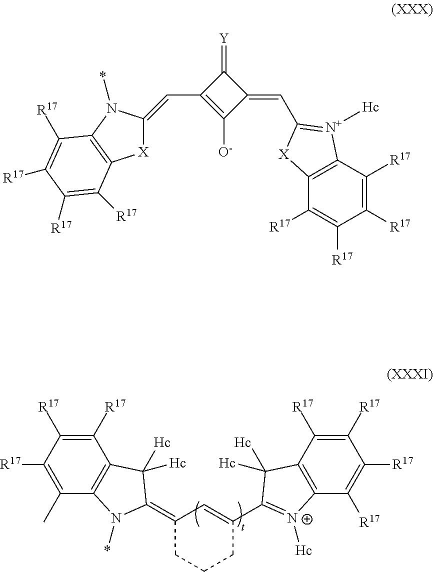

wherein Hc in each occurrence is independently a C₁₋₂₀ hydrocarbyl group and X is O, S or CR¹⁵₂ wherein R¹⁵ in each occurrence is independently H or a C₁₋₂₀ hydrocarbyl group; Y is O, S or C(CN)₂; t is 1, 2 or 3; R¹⁷ in each occurrence is H or a substituent and R¹⁷ groups linked to adjacent carbon atoms may be linked to form an aromatic or non-aromatic ring; and * represents a bond to a host repeat unit or an intermediate repeat unit or a divalent linking group L between the emissive unit and the host repeat unit or intermediate repeat unit.

It will be understood that the dotted bonds represent optional groups which, together with the atoms they are linked to, may form a cyclohexyl ring. The cyclohexyl ring may be unsubstituted or substituted with one or more substituents, e.g. one or more groups Hc.

Optionally, the host repeat units comprise more than 50 mol % of the repeat units of the polymer.

Optionally, the host repeat units comprise 70-90 mol % of the repeat units of the polymer.

Optionally, the intermediate repeat units comprise 5-20 mol % of the repeat units of the polymer.

Optionally, the emissive repeat units comprise 3-10 mol % of the repeat units of the polymer.

In some embodiments, the present disclosure provides a light-emitting particle comprising the conjugated light-emitting polymer described herein.

Optionally, the particle comprises a matrix material.

Optionally, the matrix material is silica.

In some embodiments, the present disclosure provides a light-emitting marker comprising the conjugated light-emitting polymer or the light-emitting particle according to any one of the preceding claims and a binding group comprising a biomolecule.

In some embodiments, the present disclosure provides a precursor of the light-emitting marker described herein, the precursor comprising a functional group capable of binding to the biomolecule.

Optionally, the functional group of the precursor comprises biotin.

In some embodiments, the present disclosure provides a method of forming the light-emitting marker described herein, the method comprising binding the biomolecule to the functional group of the precursor described herein.

In some embodiments, the present disclosure provides a formulation comprising the light-emitting polymer, light-emitting particle or precursor as described herein dissolved or dispersed in one or more solvents.

In some embodiments, the present disclosure provides a method of identifying a target analyte in a sample, the method comprising irradiating the sample to which has been added a light-emitting marker as described herein and which is configured to bind to the target analyte; and detecting emission from the light-emitting marker,

Optionally, the sample comprises at least one further light-emitting marker.

Optionally, the method is a flow cytometry method and the target analyte is a target cell.

### DESCRIPTION OF THE DRAWINGS

The disclosed technology and accompanying figures describe some implementations of the disclosed technology.

FIG. 1 illustrates a flow cytometer for use in a method according to some embodiments of the present disclosure

FIG. 2 shows emission spectra in methanol solution for a polymer according to an embodiment of the present disclosure and a comparative polymer; and

FIG. 3 shows emission spectra for the polymers of FIG. 2 in nanoparticles.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that conjugated light-emitting polymers containing a host repeat unit, an emissive unit and an intermediate repeat unit having a band gap intermediate between that of the host repeat unit and the emissive unit may provide bright light emission upon arrangement of the emissive unit in close proximity to the intermediate repeat unit.

The emissive unit is either:
an emissive repeat unit of a block copolymer wherein the block copolymer comprises a first block comprising the intermediate repeat unit and the emissive repeat unit and a second block comprising the host repeat unit;
a repeat unit in the polymer backbone wherein at least some, optionally all, of the emissive repeat units are arranged so as to be directly adjacent to an intermediate repeat unit;
a substituent pendant from the intermediate repeat unit; or
a substituent pendant from a host repeat unit wherein at least some, optionally all, of the host repeat units substituted with an emissive unit are arranged so as to be directly adjacent to an intermediate repeat unit.

By “conjugated light-emitting polymer” as used herein is meant a polymer having a backbone containing repeat units that are directly conjugated to adjacent repeat units in the polymer backbone.

In use, the polymer is irradiated and light may be absorbed by the host repeat unit. Energy absorbed by the host repeat unit may be transferred to the emissive repeat unit via the intermediate repeat unit. Arrangement of the emissive unit in close proximity to the intermediate repeat unit may facilitate energy transfer from the intermediate repeat unit to the emissive repeat unit.

Preferably, there is an overlap between the emission spectrum of the conjugated light-emitting polymer in which the emissive unit is absent, and the absorption spectrum of the emissive unit. More preferably, at least part of the width of the half maximum of an emission peak of the conjugated light-emitting polymer in which the emissive unit is absent overlap an absorption peak of the emissive unit.

Optionally, the emissive unit has a peak emission wavelength of at least 500 nm, optionally in the range of 500 nm to 850 nm. Optionally, the emissive unit has a full width at half maximum (FWHM) of less than 100 nm, preferably less than 50 nm.

Optionally, the Stokes shift of the conjugated light-emitting polymer is greater than 100 nm.

Optionally, the Stokes shift of the conjugated light-emitting polymer in which the emissive unit is absent is less than 100 nm.

Preferably, the host repeat units comprise more than 50 mol % of the repeat units of the polymer, optionally 70-90 mol %.

Preferably, the intermediate repeat units comprise 5-20 mol % of the repeat units of the polymer.

Preferably, the emissive repeat units, which may be emissive repeat units in the polymer backbone or repeat units substituted with an emissive unit, comprise 3-10 mol %, optionally 5-7 mol % of the repeat units of the polymer.

In some embodiments, the emissive unit is pendant from an intermediate repeat unit. In these embodiments, some or all of the intermediate repeat units of the conjugated light-emitting polymer are substituted with the emissive unit.

In some embodiments, the emissive unit is pendant from a host repeat unit. In these embodiments, some of the host repeat units of the conjugated light-emitting polymer are substituted with the emissive unit and the remaining host repeat units are not substituted with an emissive group.

The polystyrene-equivalent number-average molecular weight (Mn) measured by gel permeation chromatography of the polymers described herein, preferably the light-emitting polymers described herein may be in the range of about 5×10³ to 1×10⁸, and preferably 1×10⁴ to 5×10⁶. The polystyrene-equivalent weight-average molecular weight (Mw) of the polymers described herein may be 1×10³ to 1×10⁸, preferably 1×10⁴ to 1×10⁷, more preferably 3×10⁴ to 5×10⁴.

Host Repeat Unit

The band gap of the host repeat unit is wider than that of the intermediate repeat unit. For the purpose of this comparison, the band gap of the host repeat unit and intermediate repeat unit may be taken to be the band gaps of the respective monomers for forming these repeat units.

It will be understood that conjugation of the host repeat unit and intermediate repeat units to adjacent repeat units in the polymer may reduce band gaps of these repeat units in the polymer as compared to the band gaps of the corresponding monomer.

The host repeat unit may be an arylene repeat unit; a repeat unit of formula (I); or a repeat unit of formula (II). Each host repeat unit as described herein is unsubstituted or substituted with one or more substituents.

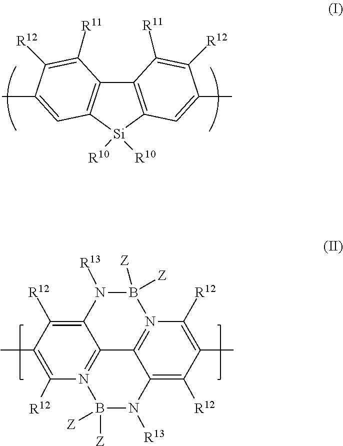

wherein R¹⁰ in each occurrence is independently a substituent; R¹¹ in each occurrence is independently H or a substituent and two R¹¹ groups may be linked to form a ring; R¹² independently in each occurrence is H or a substituent; R¹³ independently in each occurrence is a C₁₋₂₀ hydrocarbyl group; and Z in each occurrence is independently a substituent.

An arylene host repeat unit is optionally a C₆-C₁₄ arylene repeat unit, for example a repeat unit selected from phenylene, fluorene, benzofluorene, phenanthrene, dihydrophenanthrene, naphthalene or anthracene.

Arylene repeat units may be selected from repeat units of formulae (III)-(VT):

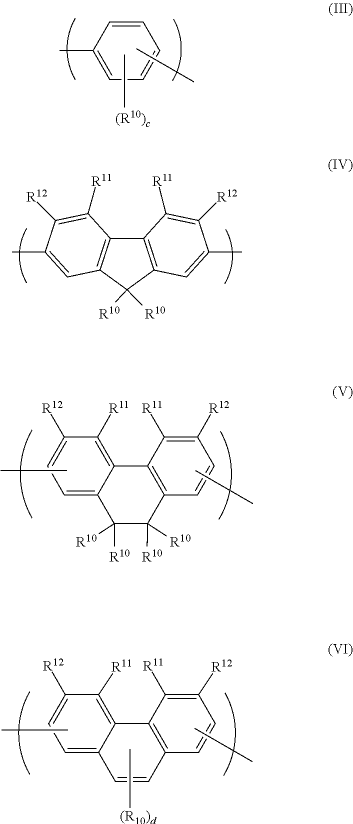

wherein R¹⁰-R¹² are as described above with reference to formulae (I) and (II); c is 0, 1, 2, 3 or 4, preferably 1 or 2; and d is 0, 1 or 2.

Optionally, each R¹⁰ is independently selected from:
C₁₋₂₀ alkyl wherein one or more non-adjacent, non-terminal C atoms may be replaced with O, S, COO or CO and one or more H atoms of the alkyl may be replaced with F;
a group of formula —(Ar²)p wherein Ar² in each occurrence is independently an aryl or heteroaryl group, preferably phenyl, which is unsubstituted or substituted with one or more substituents;
a group comprising the emissive unit; and
an ionic substituent.

By “non-terminal C atom” of an alkyl group as used herein means a C atom other than the methyl group at the end of an n-alkyl chain or the methyl groups at the ends of a branched alkyl chain.

In the case where R¹⁰ is a group comprising the emissive unit, the emissive unit may be directly bound to the arylene unit or it may be linked to the arylene unit by a divalent linking group L. Optionally, the divalent linking group L is selected from linear or branched C₁₋₂₀ alkylene, more preferably C₁₋₁₀ alkylene, wherein one or more non-adjacent C atoms may be replaced with O, S, NR¹³, Si(R¹³)₂, CO, COO, CON(R¹³) or phenylene wherein R¹³ in each occurrence is independently a C₁₋₂₀ hydrocarbyl group.

Preferably, each R¹¹ is H or both R¹¹ groups are linked to form a ring, optionally a 6 or 7 membered ring. Optionally, two R¹¹ groups are linked to form a ring in which the linked R¹¹ groups form a C₂- or C₃-alkylene chain wherein one or more non-adjacent C atoms of the alkylene chain may be replaced with O, S, NR¹³ or Si(R¹³)₂ wherein R¹³ in each occurrence is independently a C₁₋₂₀ hydrocarbyl group.

An exemplary repeat unit in which both R¹¹ groups are linked has formula (IVa):

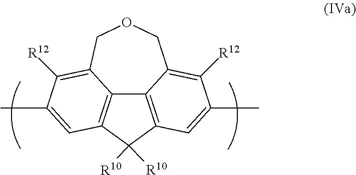

Each R¹² is preferably H or a substituent R¹⁰, more preferably H.

Preferably, Z independently in each occurrence is selected from the group consisting of branched, linear or cyclic C₁₋₂₀ alkyl; phenyl which is unsubstituted or substituted with one or more substituents, e.g. one or more C₁₋₁₂ alkyl groups; and F.

A hydrocarbyl group as described anywhere herein is optionally selected from a linear, branched or cyclic alkyl, optionally C₁₋₂₀ alkyl; unsubstituted phenyl; and phenyl substituted with one or more C₁₋₁₂ alkyl groups.

Optionally substituents of Ar², where present are selected from F, CN, NO₂ and C₁₋₂₀ alkyl wherein one or more non-adjacent, non-terminal C atoms may be replaced with O, S, COO or CO and one or more H atoms of the alkyl may be replaced with F.

Preferably, an ionic substituent as described herein has formula (VII):

-(Sp¹)ₘ—(R³)ₙ (VII)

wherein Sp¹ is a spacer group; m is 0 or 1; R³ independently in each occurrence is an ionic group; n is 1 if m is 0 and n is at least 1, optionally 1, 2, 3 or 4, if m is 1.

Preferably, Sp¹ is selected from:
C₁₋₂₀ alkylene or phenylene-C₁₋₂₀ alkylene wherein one or more non-adjacent C atoms may be replace with O, S, N or C═O;
a C₆₋₂₀ arylene or 5-20 membered heteroarylene, more preferably phenylene, which, other than the one or more ionic groups R³, may be unsubstituted or substituted with one or more non-ionic substituents, optionally one or more non-ionic substituents R⁸ as described above.

More preferably, Sp¹ is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non-ionic substituents.

In a preferred embodiment, Sp¹ is a C₆₋₂₀ arylene or 5-20 membered heteroarylene, more preferably phenylene, substituted with a group of formula (VIII):

—O(R⁴O)ᵥ—R⁵ (VIII)

wherein R⁴ in each occurrence is a C₁₋₁₀ alkylene group, optionally a C₁₋₅ alkylene group, wherein one or more non-adjacent, non-terminal C atoms of the alkylene group may be replaced with O, R⁵ is H or C₁₋₅ alkyl, and v is 0 or a positive integer, optionally 1-10. Preferably, v is at least 2. More preferably, v is 2 to 5. The value of v may be the same in all the polar groups of formula —O(R⁴O)ᵥ—R⁵. The value of v may differ between different groups of formula (VIII) of the same polymer.

Optionally, the group of formula (VIII) has formula —O(CH₂CH₂O)ᵥR⁵ wherein v is at least 1, optionally 1-10 and R⁵ is a C₁₋₅ alkyl group, preferably methyl. Preferably, v is at least 2. More preferably, v is 2 to 10.

The ionic group R³ may be anionic or cationic.

Exemplary anionic group are —COO⁻, a sulfonate group; hydroxide; sulfate; phosphate; phosphinate; or phosphonate.

An exemplary cationic group is —N(R⁶)₃⁺ wherein R⁶ in each occurrence is H or C₁₋₁₂ hydrocarbyl. Preferably, each R⁶ is a C₁₋₁₂ hydrocarbyl.

A conjugated polymer as described herein comprising cationic or anionic groups comprises counterions to balance the charge of these ionic groups. An anionic or cationic group and counterion may have the same valency, with a counterion balancing the charge of each anionic or cationic group. The anionic or cationic group may be monovalent or polyvalent. Preferably, the anionic and cationic groups are monovalent.

The conjugated polymer may comprise a plurality of anionic or cationic polar groups wherein the charge of two or more anionic or cationic groups is balanced by a single counterion. Optionally, the polar groups comprise anionic or cationic groups comprising di- or trivalent counterions.

In the case of an anionic group, the cation counterion is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group, the anion counterion is optionally a halide; a sulfonate group, optionally mesylate or tosylate; hydroxide; carboxylate; sulfate; phosphate; phosphinate; phosphonate; or borate.

Intermediate Repeat Unit

The intermediate repeat unit is any repeat unit which, upon incorporation into the backbone of the conjugated polymer, reduces the HOMO-LUMO band gap of the polymer as compared to a polymer consisting of the host repeat unit only. The polymer consisting of the host repeat unit and intermediate repeat unit has a larger band gap than that of the emissive unit.

The band gap of the intermediate repeat unit may be the band gap of the monomer for forming this repeat unit. It will be understood by the skilled person that conjugation of the intermediate repeat unit to adjacent repeat units in the polymer backbone, e.g. conjugation of the intermediate repeat unit to host repeat units, may reduce this band gap.

The intermediate repeat unit may comprise one or more 5-20 membered heteroarylene groups, each of which is optionally and independently unsubstituted or substituted with one or more substituents including, without limitation, repeat units comprising thiophene, bithiophene, benzothiadiazole, and combinations thereof.

Exemplary heteroarylene intermediate repeat units include repeat units of formulae (IX)-(XII):

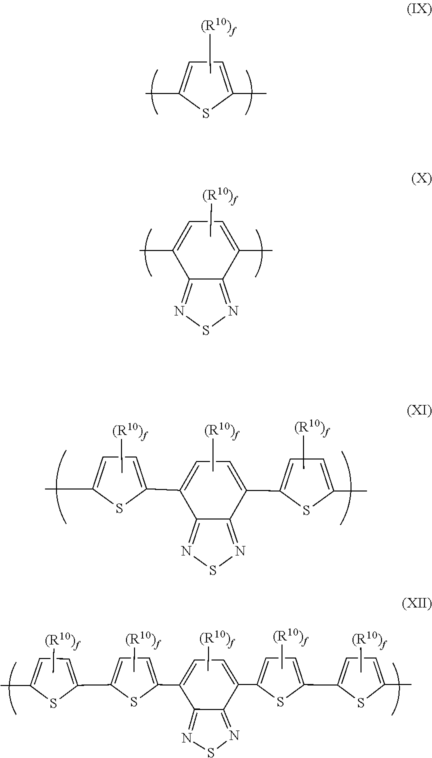

wherein R¹⁰ in each occurrence is independently a substituent as described above, and f in each occurrence is independently 0, 1 or 2.

In the case where R¹⁰ is a group comprising the emissive unit, the emissive unit may be directly bound to the intermediate repeat unit or it may be linked to the intermediate repeat unit by a divalent linking group L as described above.

The intermediate repeat unit may be amine repeat unit, optionally a repeat unit of formula (XIII):

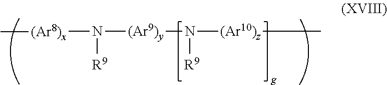

wherein Ar⁸, Ar⁹ and Ar¹⁰ in each occurrence are independently selected from substituted or unsubstituted aryl or heteroaryl, g is 0, 1 or 2, preferably 0 or 1, R⁹ independently in each occurrence is a substituent, and x, y and z are each independently 1, 2 or 3.

R⁹, which may be the same or different in each occurrence when g is 1 or 2, is preferably selected from the group consisting of an emissive group, alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl that may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, that may be unsubstituted or substituted with one or more substituents.

R⁹ is preferably Ar¹¹ or a branched or linear chain of Ar¹¹ groups. Ar¹¹ in each occurrence is preferably phenyl that may be unsubstituted or substituted with one or more substituents.

Exemplary groups R⁹ include the following, each of which may be unsubstituted or substituted with one or more substituents, and wherein * represents a point of attachment to N:

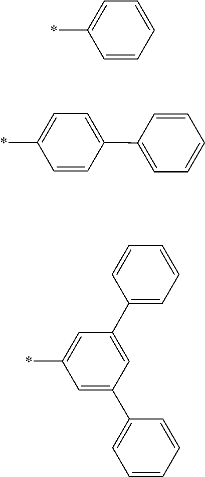 [SMILES: *c1ccccc1 ; *c1ccc(-c2ccccc2)cc1 ; *c1cc(-c2ccccc2)cc(-c2ccccc2)c1]

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be selected from groups of formula R¹⁰ as described above. Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (XVIII) include unsubstituted or substituted units of formulae XVIII-1), (XVIII-2) and (XVIII-3):

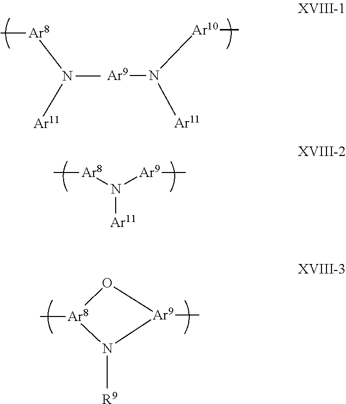

The intermediate repeat unit may be selected from formulae (XIII-XVII):

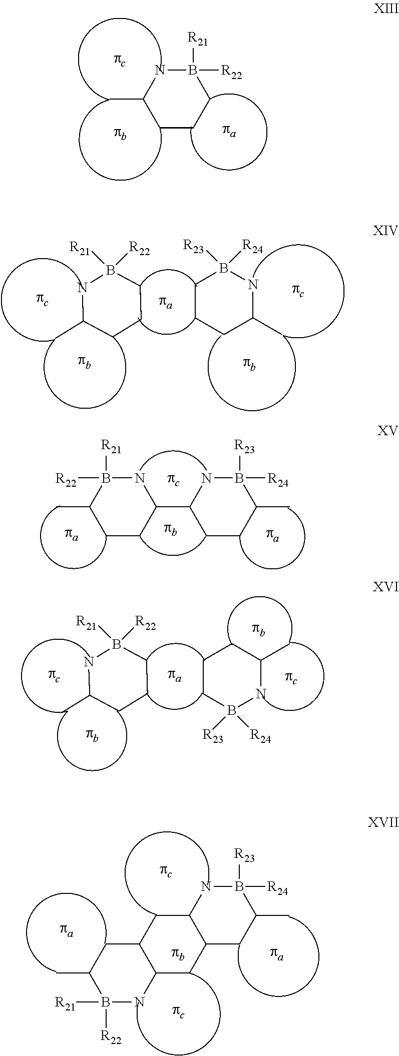

wherein:
R²¹, R²², R²³ and R²⁴ are each independently selected from H, F, Cl, Br, substituted or unsubstituted C₁₋₁₀alkyl, substituted or unsubstituted C₂₋₁₀alkenyl, substituted or unsubstituted C₂₋₁₀ alkynyl, substituted or unsubstituted aryl, substituted or unsubstituted heteroaryl, substituted or unsubstituted amino, substituted or unsubstituted C₁₋₁₀ alkoxy, substituted or unsubstituted arylalkoxy, and hydroxyl;
each πa independently represents a π-conjugated π-donor ring system formed from one, two, three or four 6-membered aryl or 5 to 6-membered heteroaryl rings; each πb independently represents a π-conjugated 6-membered aryl or 5-6 membered heteroaryl ring:
each πc independently represents a π-conjugated 5-6 membered heteroaryl ring, which when taken in combination with πb, forms a π-acceptor ring system; wherein:
any or all of the rings forming πa, πb and πc may be independently optionally substituted with one more ring substituents selected from an emissive group, halo, (1-20C)alkyl, (2-20C)alkenyl, (2-20C)alkynyl, aryl, heteroaryl, cycloalkyl, heterocyclyl, carboxyl, phosphoryl, sulfonyl, hydroxyl, (1-20C)alkoxy, nitro, amino, mercapto, silyl, siloxy, azido, boronic acid group, sulfonic acid group, hydroxamic acid group, cyanoacrylate group, and dioxocyclobutenyl group having at least one functional group selected from the group consisting of a carboxyl, phosphoryl, sulfonyl, hydroxyl, alkoxy, nitro, amino, mercapto, silyl, siloxy, azido, boronic acid group, sulfonic acid group, hydroxamic acid group and cyanoacrylate group.

The repeat unit of formula (XIII) may be bound into the polymer backbone by a bond to Ra and one of πb and πc.

The repeat units of formula (XIV) and (XVI) may be bound into the polymer backbone by a bond to πb or πc on one side of πa and by a bond to πb or πc on the other side of πa.

The repeat unit of formula (XV) may be bound into the polymer backbone by a bond to each one of πa.

The repeat unit of formula (XVII) may be bound into the polymer backbone by a bond to each one of πa or by a bond to each one of πc.

Exemplary repeat units of formulae (XIII)-(XVII) include:

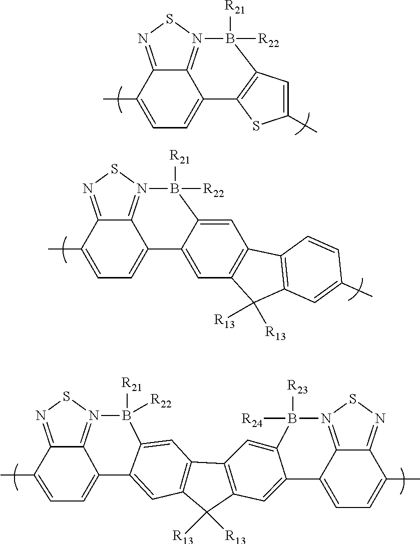

In some embodiments, the conjugated light-emitting polymer contains only one intermediate repeat unit. In these embodiments, the conjugated light-emitting polymer may contain only one host repeat unit or two or more different host repeat units, e.g. two or more different arylene repeat units as described herein.

In some embodiments, the conjugated polymer contains two or more different intermediate repeat units, e.g. two or more different repeat units of formula (IX)-(XVIII) as described herein. The two or more different intermediate repeat units preferably each have a band gap that is different from any other of the intermediate repeat units. In these embodiments, the conjugated light-emitting polymer may contain only one host repeat unit or two or more different host repeat units, e.g. two or more different arylene repeat units as described herein.

Emissive Unit

The conjugated polymer emits light of peak wavelength λ_{E}. The emissive unit, upon incorporation into the polymer, may increase the peak emission wavelength to λ_{E} as compared to a polymer consisting of the host repeat unit and intermediate repeat unit only.

The emissive unit has a smaller band gap than the intermediate repeat unit.

The band gap of an emissive unit may be the band gap of a monomer for forming an emissive repeat unit, which may either be an emissive repeat unit in the polymer backbone or a repeat unit having a pendant emissive unit. In the case where the emissive unit is a repeat unit conjugated to adjacent repeat units, it will be understood by the skilled person that peak wavelength λ_{E} may be longer than that of the monomer of the emissive repeat unit due to conjugation of the emissive repeat unit to adjacent repeat units, e.g. due to conjugation of the emissive repeat unit to adjacent host repeat units.

In the case where the emissive unit is pendant from the polymer backbone, in some embodiments the emissive unit is pendant from an intermediate repeat unit. If the polymer contains more than one intermediate repeat unit, the emissive unit is preferably pendant from the lowest band gap intermediate repeat unit.

In the case where the emissive unit is pendant from the polymer backbone, in some embodiments the emissive unit is pendant from a host repeat unit which is adjacent to an intermediate repeat unit. If the polymer contains more than one intermediate repeat unit, the emissive unit is preferably pendant from an arylene repeat unit arranged adjacent to the lowest band gap intermediate repeat unit.

In some embodiments, the conjugated light-emitting polymer comprises a repeat unit selected from formulae (XX), (XXI) and (XXII):

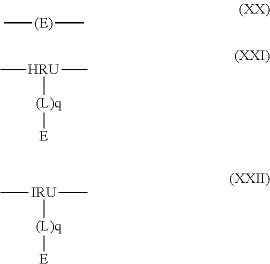

wherein E is an emissive unit; HRU is a host repeat unit; IRU is an intermediate repeat unit; L is a divalent linker group; and q is 0 or 1.

Optionally, L is selected from linear or branched C₁₋₂₀ alkylene, more preferably C₁₋₁₀ alkylene, wherein one or more non-adjacent C atoms may be replaced with O, S, NR¹³, Si(R¹³)₂, CO, COO, CON(R¹³) or phenylene wherein R¹³ in each occurrence is independently a C₁₋₂₀ hydrocarbyl group.

In the case where the conjugated polymer comprises an emissive repeat unit in the backbone of the polymer, the polymer is preferably a block copolymer having a first block containing the emissive repeat unit and the intermediate repeat unit and a second block containing the host repeat unit. More efficient energy transfer may be achieved by providing the emissive repeat unit and the intermediate repeat unit in the same block as compared to a polymer in which the emissive and intermediate repeat units are evenly distributed throughout the polymer backbone, particularly if a relatively low proportion of the repeat units are emissive and intermediate repeat units.

In some embodiments, the repeat units of the first block consist of the intermediate repeat unit and the emissive repeat unit, e.g. in an alternating arrangement.

In some embodiments, the repeat units of the first block comprise or consist of the intermediate repeat unit, the emissive repeat unit and the host repeat unit. In these embodiments, there is preferably no more than a single host repeat unit separating an intermediate or host repeat unit from another intermediate or host repeat unit.

Preferably, the block copolymer comprises a second block in which more than 80 mol % of the repeat units are host repeat units. More preferably at least 90 mol % and most preferably 100% of the repeat units of the second block are host repeat units.

Exemplary emissive units include:

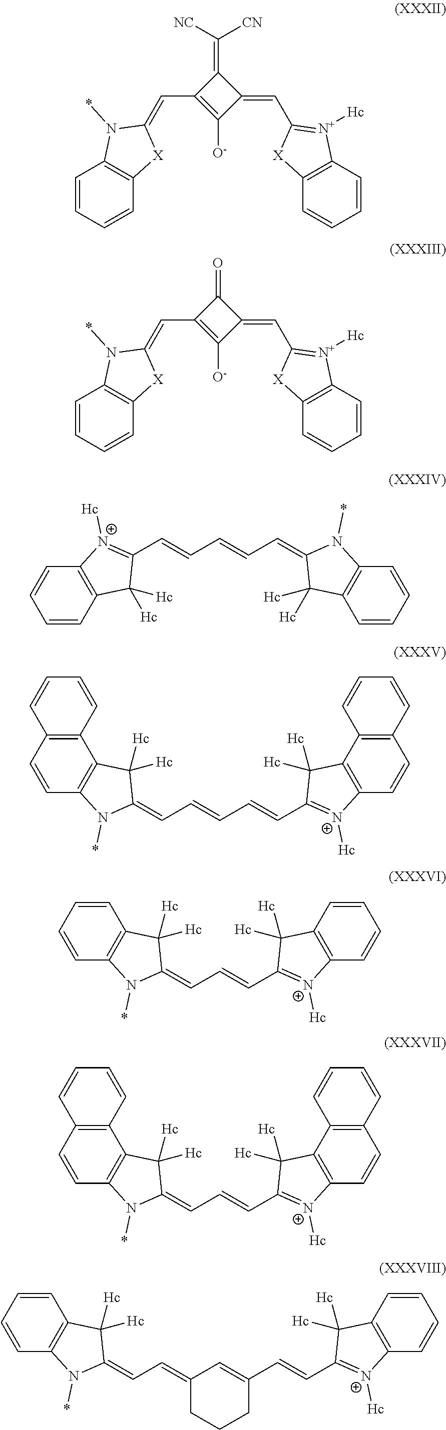

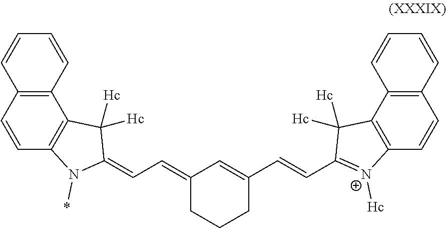

wherein Hc in each occurrence is independently a C₁₋₂₀ hydrocarbyl group, optionally a C₁₋₂₀ alkyl group and X is O, S or CR¹⁵₂ wherein R¹⁵ in each occurrence is independently H or a C₁₋₂₀ hydrocarbyl group, preferably H or C₁₋₂₀ alkyl; and * represents a bond to L or to a host repeat unit or an intermediate repeat unit.

The charge of cationic groups (XXXIII)-(XXXIX) may be balanced by any suitable anion, e.g. a halide or pseudohalide anion.

It will be understood that the width of the band gap of the emissive unit will depend on the width of the intermediate repeat unit band gap. Accordingly, any of the repeat units described herein as intermediate repeat units may, if used with another intermediate repeat unit having a wider band gap, be used as emissive repeat units. For example, a repeat unit of formula (XI) or (XII) may be used as an emissive repeat unit in combination with an intermediate repeat unit of formula (X). Exemplary host or intermediate repeat units carrying an emissive unit include the following:

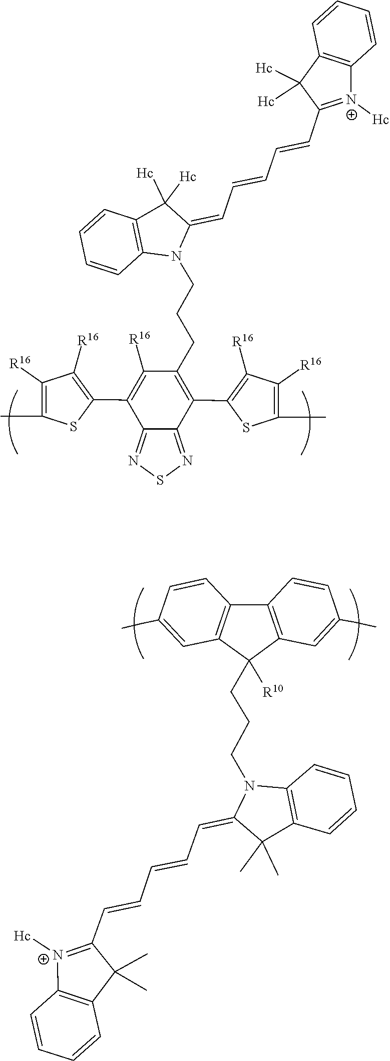

wherein R¹⁶ is H or a substituent R¹⁰ as described above.

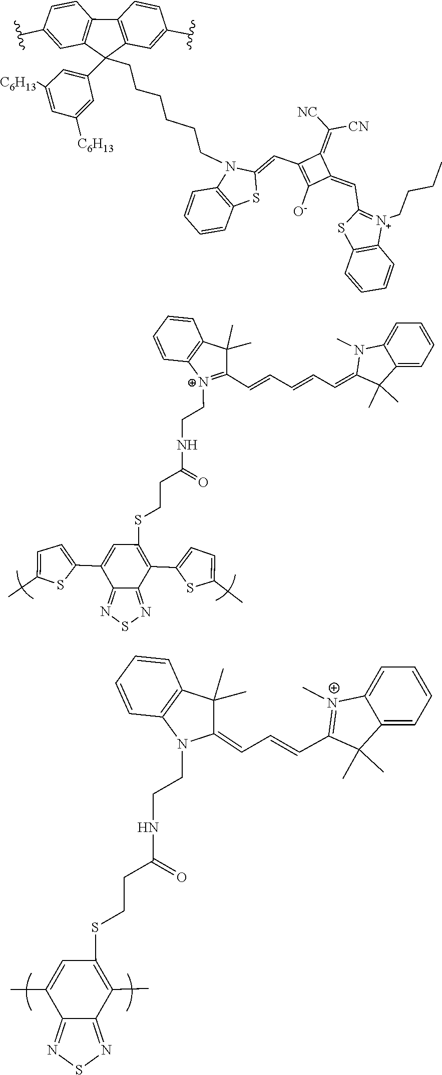

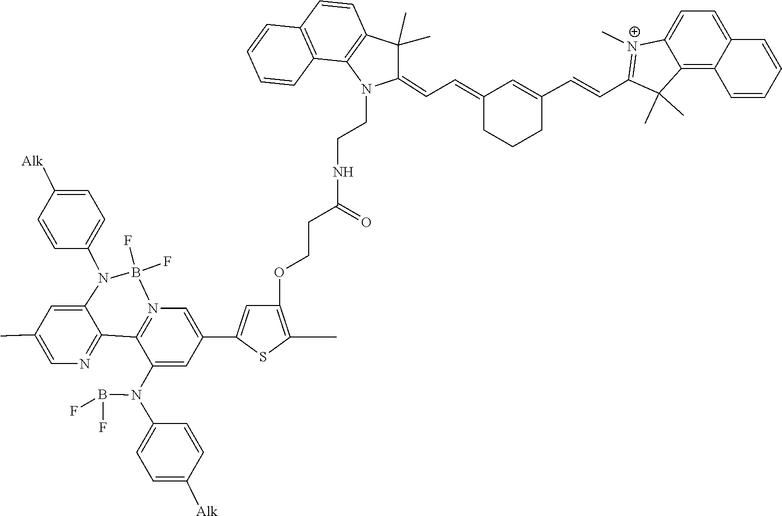

wherein Alk is a C₁₋₁₂ alkyl group.

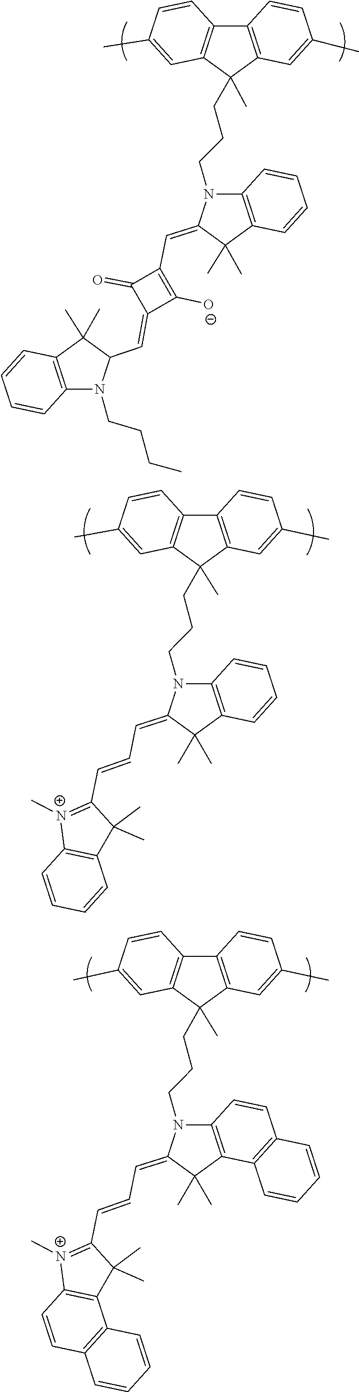

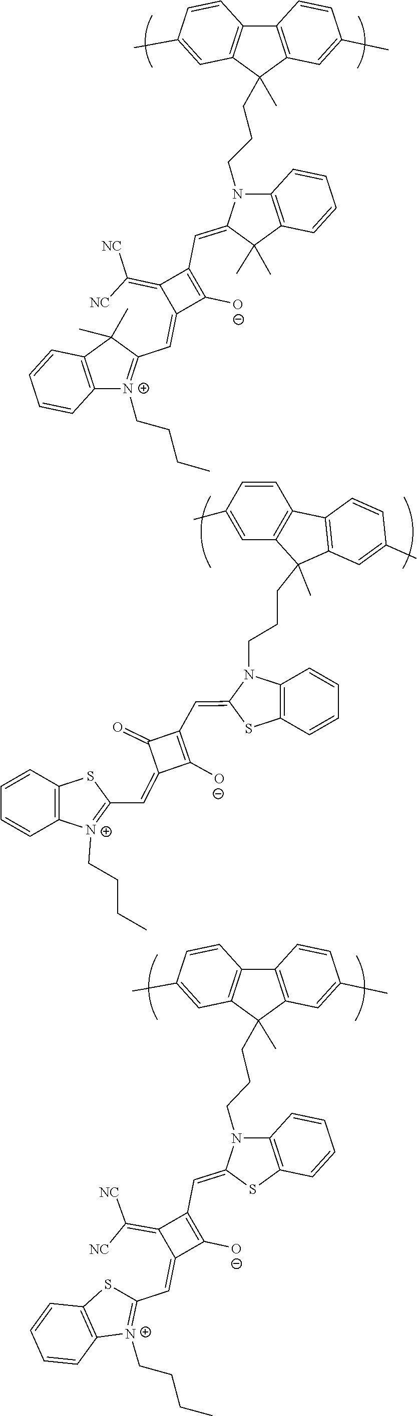

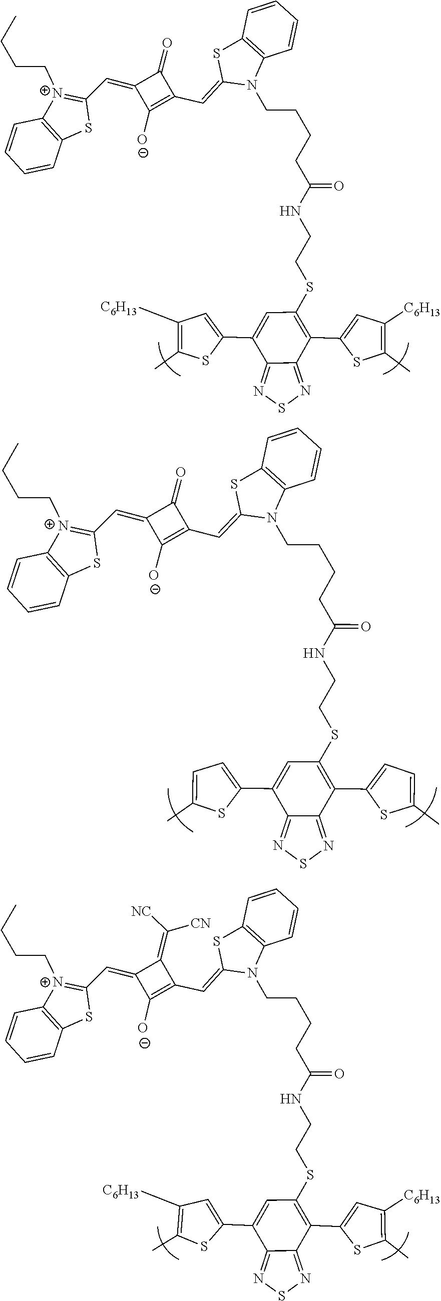

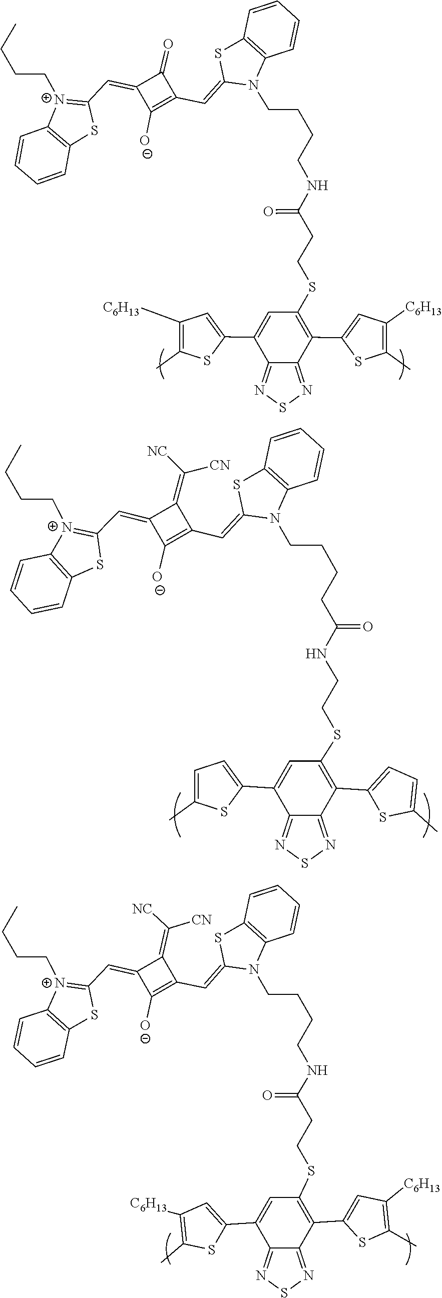

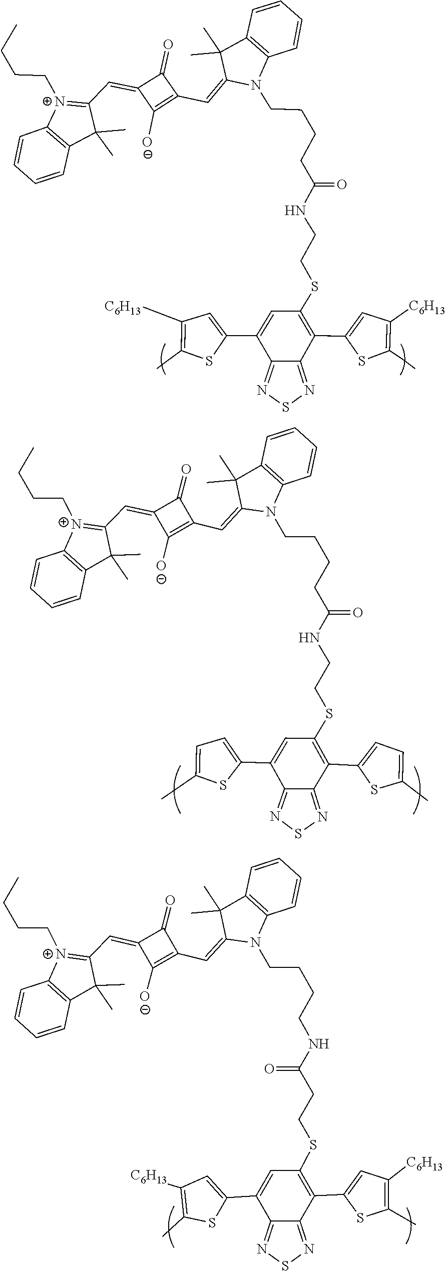

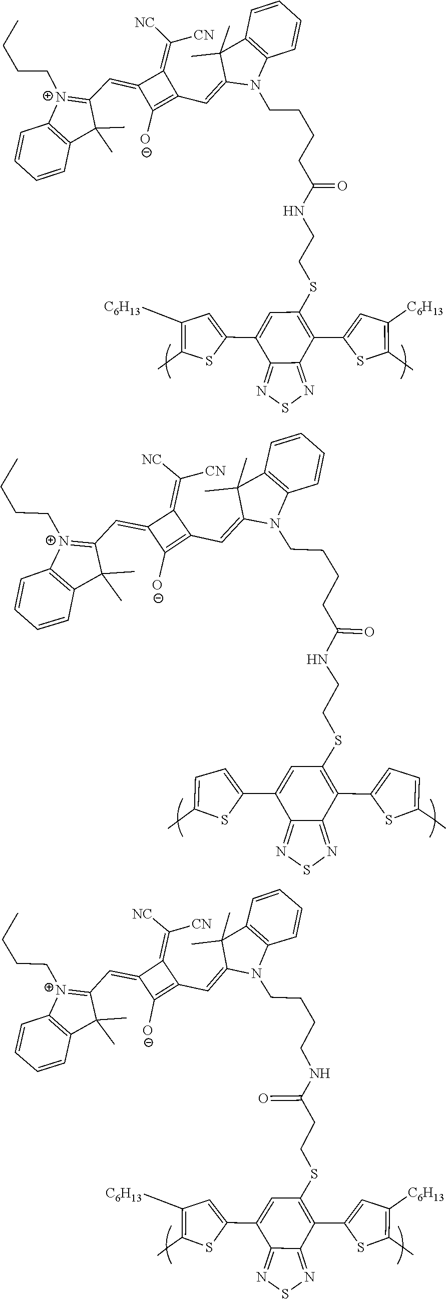

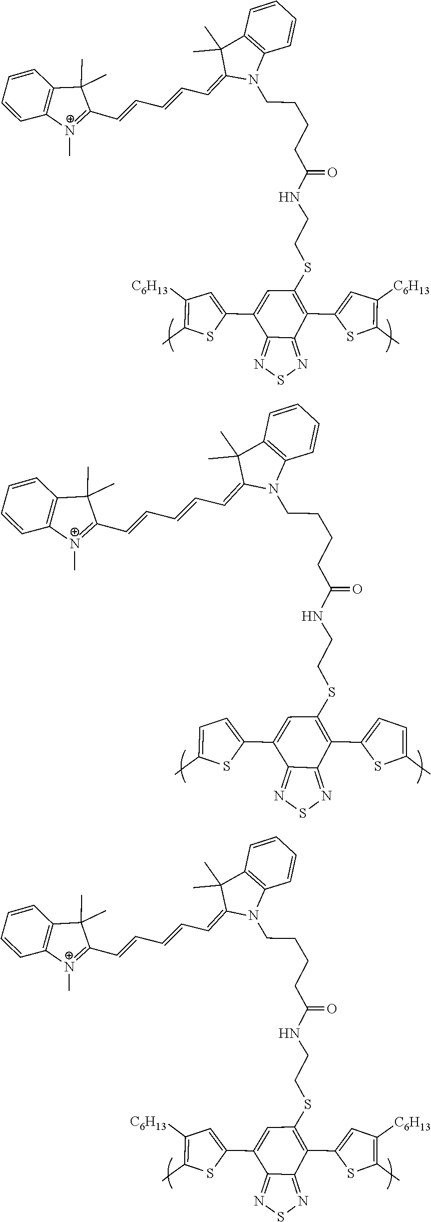

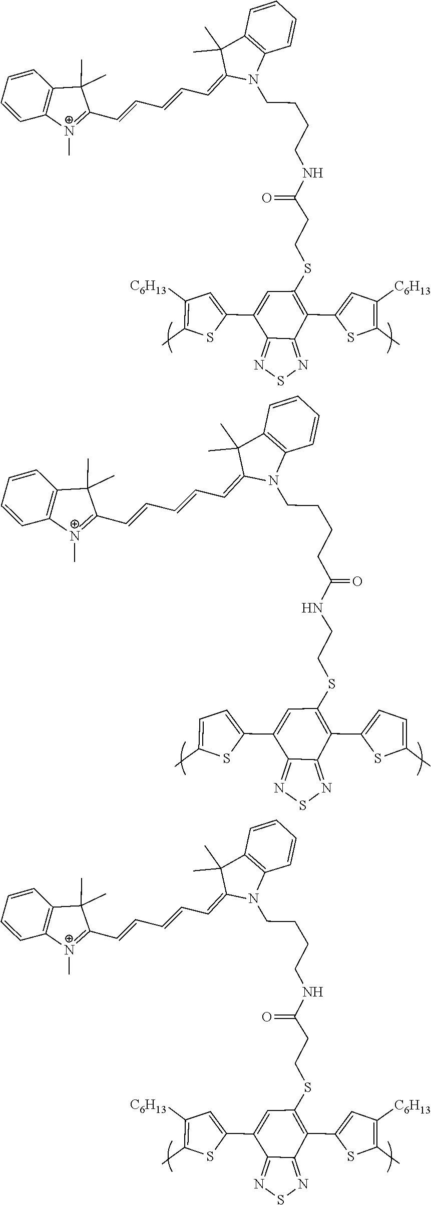

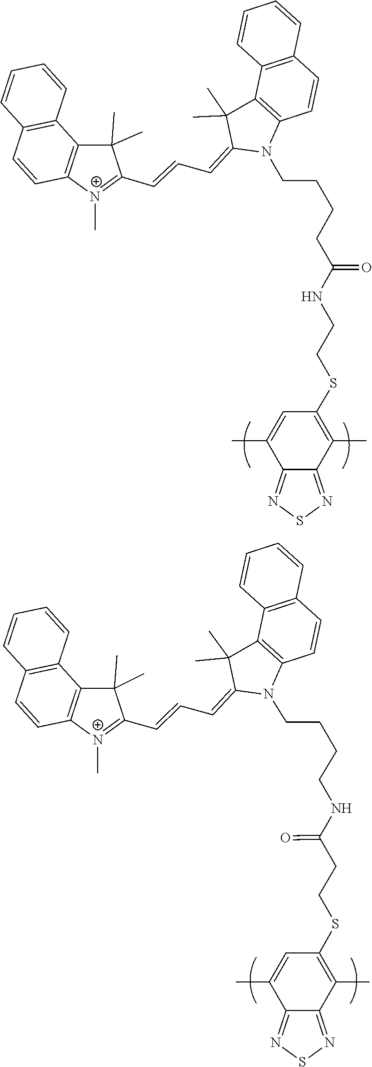

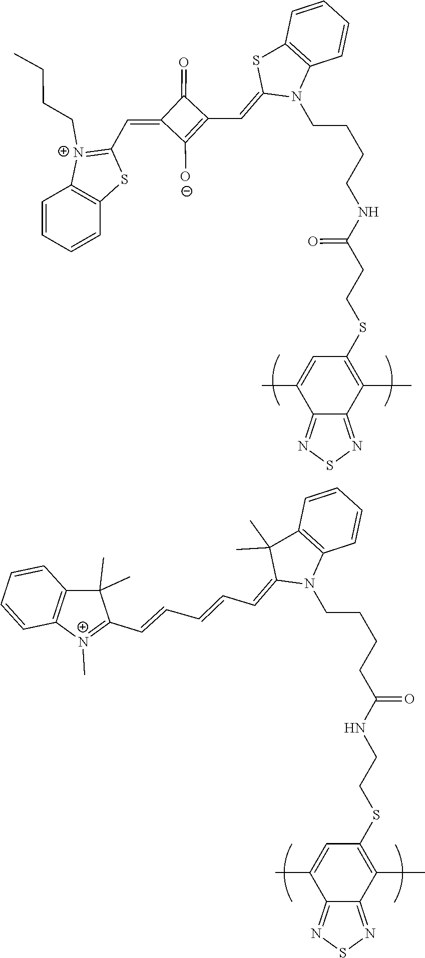

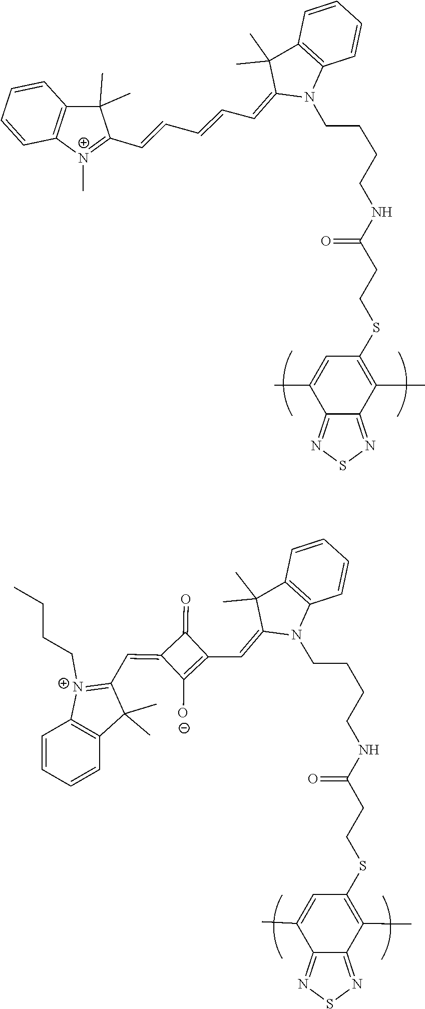

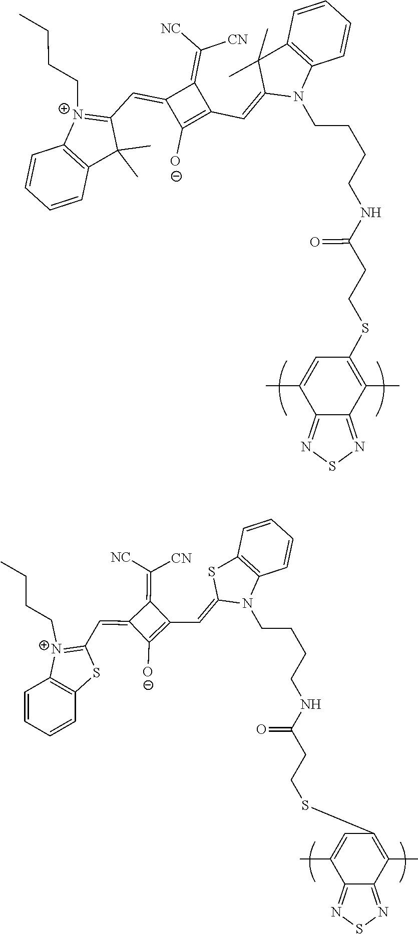

Exemplary first blocks of a block copolymer containing intermediate and emissive repeat units include, without limitation:

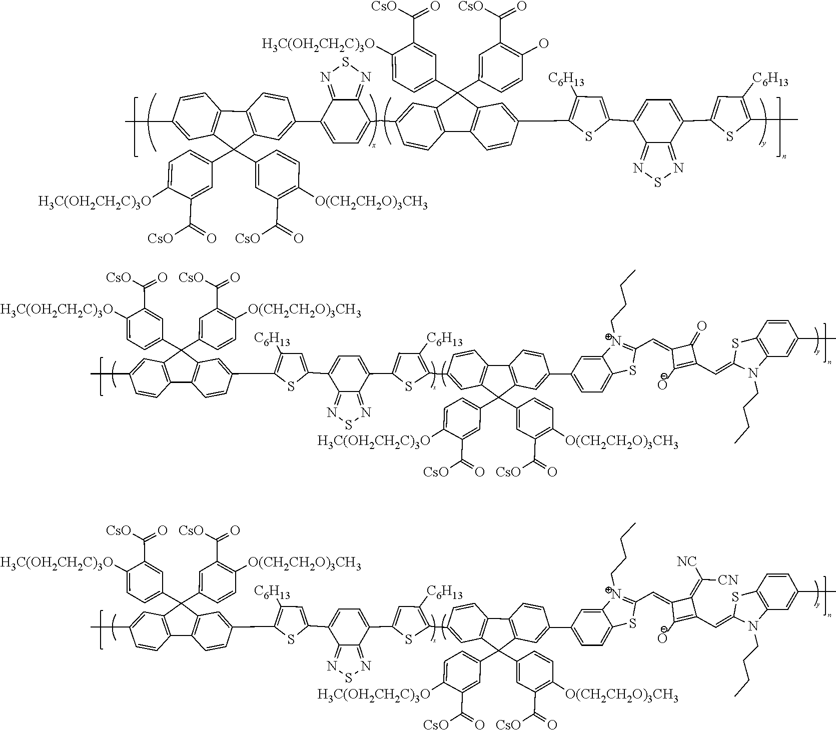

Polymer Formation

The conjugated light-emitting polymers described herein may be formed by any method known to the skilled person. Arrangement of repeat units within the polymer backbone may be controlled by, e.g. formation of block copolymers, use of polymerisation methods requiring monomers with different reactive groups; and selection of monomer ratio.

Conjugated polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

The monomers may be formed by polymerisation of monomers containing boronic acid leaving groups or esters thereof, and halide or pseudohalide (e.g. sulfonate) leaving groups. The skilled person will understand that leaving groups may be selected to control which monomers may or may not form adjacent repeat units in the polymer.

In some embodiments, formation of the light-emitting polymer comprises polymerisation of monomers including one or more host monomers, one or more intermediate monomers and one or more emissive monomers for forming host repeat units, intermediate repeat units and emissive repeat units, respectively, in the polymer backbone. Preferably, the light-emitting polymer according to these embodiments is a block copolymer in which a first block is formed by polymerisation of the intermediate monomer, the emissive monomer and, optionally some host monomer and a second block is formed by polymerisation of the remaining monomers. Preferably, all of the intermediate monomer and the emissive monomer is polymerised into the first block, optionally with some but not all of the host monomer. Preferably, the host monomer is the only monomer present in formation of the second block.

In some embodiments, monomers for formation of the second block are reacted with the first block. In some embodiments, the first and second blocks are formed separately and then combined.

In some embodiments, formation of the light-emitting polymer comprises polymerisation of monomers including one or more host monomers and one or more intermediate monomers for forming host repeat units and intermediate repeat units, respectively, in the polymer backbone wherein the monomers include a host monomer or an intermediate monomer having the emissive unit pendant therefrom.

In some embodiments, the monomers include a monomer, optionally a host monomer or an intermediate monomer, having a first reactive substituent group. Following polymerisation, repeat units substituted with a reactive substituent group may be reacted with a material comprising the emissive unit and a second reactive substituent group wherein the first and second reactive substituent groups react to bind the emissive unit to the polymer backbone.

In some embodiments, the first reactive substituent group is one of an alkene and a thiol and the second reactive substituent group is the other of an alkene and a thiol, wherein the first and second reactive substituent groups react to form a linker group comprising a thioether linking the emissive unit to the polymer backbone.

In some embodiments, the first reactive substituent group is a carboxylic acid or ester, chloride or anhydride thereof, e.g. an NHS ester, and the second reactive substituent group is an alcohol or an amine, wherein the first and second reactive substituent groups react to form a linker group comprising an ester or amide linking the emissive unit to the polymer backbone.

Light-Emitting Marker

A light-emitting marker for detection of a target analyte may comprise a conjugated light-emitting polymer as described herein.

In some embodiments, a binding group having affinity for a target analyte is bound, preferably covalently bound, to the conjugated light-emitting polymer. The binding group may be provided as a side group of a repeat unit of the light-emitting polymer or as an end-group of the light-emitting polymer. In some embodiments, the conjugated light-emitting polymer in use, e.g. in flow cytometry, may be dissolved or dispersed in a sample to be analysed. In the case where it is dissolved, the conjugated light-emitting polymer is preferably dissolved in water.

In some embodiments, the light-emitting marker is a particulate marker. In use, e.g. during flow cytometry, the particulate light-emitting marker may be dispersed in a sample to be analysed.

In some embodiments, the light-emitting marker particles comprise the conjugated light-emitting polymer in collapsed form.

In preferred embodiments, the light-emitting marker particles comprise a matrix material and the conjugated light-emitting polymer. The matrix material is preferably an inorganic matrix material, e.g. silica. According to these embodiments, the binding group may be bound, preferably covalently bound, to the matrix.

Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

Light-emitting marker particles may comprise a core and, optionally, one or more shells surrounding the core.

Polymer chains of the conjugated light-emitting polymer may extend across some or all of the thickness of the core and/or shell. Polymer chains may be contained within the core and/or shell or may protrude through the surface of the core and/or shell.

The conjugated light-emitting polymer may be mixed with the matrix material.

The conjugated light-emitting polymer may be bound, e.g. covalently bound, to the matrix material.

In some embodiments, the particle core may be formed by polymerisation of a silica monomer in the presence of the light-emitting polymer, for example as described in WO 2018/060722, the contents of which are incorporated herein by reference.

In some embodiments, the particle core comprises a core which comprises or consists of the light-emitting polymer and at least one shell surrounding the inner core. The at least one shell may be silica.

Optionally, at least 0.1 wt % of total weight of the particle core consists of the conjugated light-emitting polymer. Preferably at least 1, 10, 25 wt % of the total weight of the particle core consists of the conjugated light-emitting polymer.

Optionally at least 50 wt % of the total weight of the particle core consists of the matrix material. Preferably at least 60, 70, 80, 90, 95, 98, 99, 99.5, 99.9 wt % of the total weight of the particle core consists of the matrix material.

The particle core as described herein is the light-emitting particle without any surface groups, e.g. binding groups or solubilising groups, thereon.

In one embodiment of the present disclosure, at least 70 wt % of the total weight of the particle core consists of the conjugated light-emitting polymer and silica. Preferably at least 80, 90, 95, 98, 99, 99.5, 99.9 wt % of the total weight of the particle core consists of the conjugated light-emitting polymer and silica. More preferably the particle core consists essentially of the conjugated light-emitting polymer and silica.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS. Preferably the particles have a number average diameter of between 5-5000 nm, optionally 10-1000 nm, preferably between 10-500 nm, most preferably between 10-100 nm as measured by a Malvern Zetasizer Nano ZS.

Surface groups may be bound to a surface of the light-emitting particles. Exemplary surface groups include, without limitation, ether-containing groups, e.g. groups containing poly(ethyleneglycol) (PEG) chains and groups containing a binding group comprising a biomolecule.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting markers are light-emitting particles dispersed in the liquid. In some embodiments, one or more of the light-emitting markers is in particle form dispersed in the liquid and one or more of the light-emitting markers is dissolved in the liquid.

The liquid may be a solution comprising salts dissolved therein, optionally a buffer solution.

In some embodiments, the particles may be stored in a powder form, optionally in a lyophilised or frozen form.

Functional Groups

The binding group of the light-emitting marker for binding to a target analyte may be attached to the light-emitting marker by attachment to a functional group of a precursor of the light-emitting marker.

In some embodiments, the functional group is covalently bound to the conjugated light-emitting polymer.

In some embodiments, the functional group is covalently bound to a matrix material of a particulate marker precursor comprising the matrix material and the conjugated light-emitting polymer.

Optionally the functional group is selected from:
amine groups, optionally —NR⁹₂ wherein R⁹ in each occurrence is independently H or a substituent, preferably H or a C₁₋₅ alkyl, more preferably H;
carboxylic acid or a derivative thereof, for example an anhydride, acid chloride or ester, acid chloride, acid anhydride or amide group;
—OH; —SH; an alkene; an alkyne; and an azide; and
biotin or a biotin-protein conjugate.

The functional group may be reacted with a biomolecule to form a linking group linking the biomolecule to the rest of the light-emitting marker, the linking group being selected from esters, amides, urea, thiourea, Schiff bases, a primary amine (C—N) bond, a maleimide-thiol adduct or a triazole formed by the cycloaddition of an azide and an alkyne.

Exemplary binding group biomolecules for binding to a target analyte include, without limitation, DNA, RNA, peptides, carbohydrates, antibodies, antigens, enzymes, proteins, hormones and combinations thereof.

In the case where the functional group is biotin, it may be conjugated to a protein, e.g. avidin, streptavidin, neutravidin and recombinant variants thereof, and a biotinylated biomolecule may be conjugated to the protein to form the light-emitting marker.

The biotinylated biomolecule may comprise an antigen binding fragment, e.g. an antibody, which may be selected according to a target antigen.

In the case of a light-emitting particle, the functional group may be bound to a surface of the light-emitting particle, e.g. bound to a matrix material of the light-emitting particle. Each functional group may be directly bound to the surface of a light-emitting particle or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a functional group. Optionally, a silica-containing particle is reacted with a siloxane.

Applications

Light-emitting markers as described herein may be used as luminescent probes for detecting or labelling a biomolecule or a cell. In some embodiments, the particles may be used as a luminescent probe in an immunoassay such as a lateral flow or solid state immunoassay. Optionally the particles are for use in fluorescence microscopy, flow cytometry, next generation sequencing, in-vivo imaging, or any other application where a light-emitting marker is brought into contact with a sample to be analysed. The analysis may be performed using time-resolved spectroscopy. The applications can medical, veterinary, agricultural or environmental applications whether involving patients (where applicable) or for research purposes.

In use the binding group of the light-emitting markers may bind to target biomolecules which include without limitation DNA, RNA, peptides, carbohydrates, antibodies, antigens, enzymes, proteins and hormones. The target biomolecule may or may not be a biomolecule, e.g. a protein, at a surface of a cell.

A sample to be analysed may brought into contact with the light-emitting marker, for example the light-emitting marker dissolved in a solution or a particulate light-emitting marker in a colloidal suspension.

In some embodiments, the sample is analysed by flow cytometry. In flow cytometry, the light-emitting marker or markers are irradiated by at least one wavelength of light, optionally two or more different wavelengths, e.g. one or more wavelengths including at least one of about 355, 405, 488, 530, 561 and 640 nm, each of which may be ±10 nm. Light emitted by the light-emitting marker(s) may be collected by one or more detectors. To provide a background signal for calculation of a staining index, measurement may be made of a light-emitting marker mixed with cells which do not bind to the light-emitting marker.

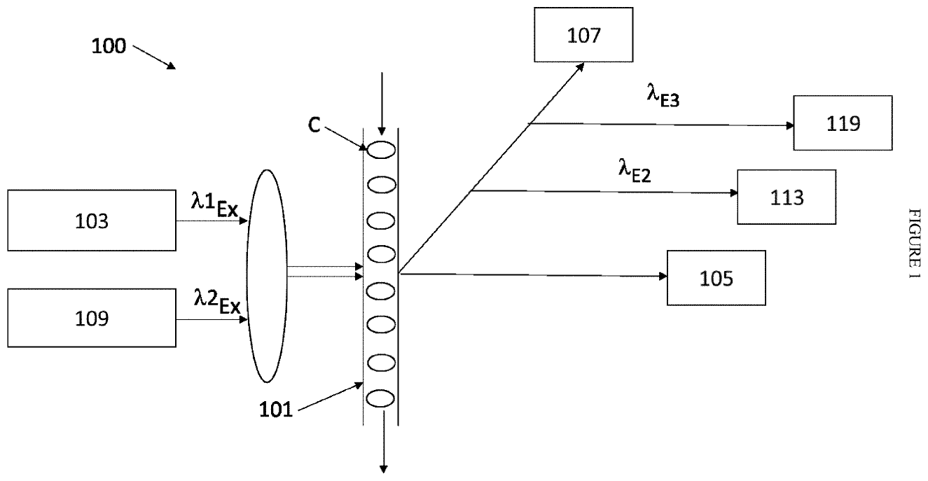

FIG. 1 schematically illustrates flow cytometer 100 which may be used in some embodiments of the flow cytometry methods of the present disclosure.

The flow cytometer comprises a flow channel 101 through which cells may pass in a single file; a first light source 103, e.g. a laser, configured to irradiate the flow channel with light of a first excitation wavelength λ1Ex; a forward scatter detector 105; a side scatter detector 107; and a first photodetector 113 configured to detect light of wavelength λE2 of a first light-emitting marker comprising a conjugated polymer as described herein bound to a cell upon excitation by the first excitation wavelength λ1Ex.

The apparatus may further comprise at least one further light source 105, e.g. a laser, configured to irradiate the flow channel with light of a second excitation wavelength λ2Ex and a photodetector configured to detect light of a second emission wavelength λE3 emitted from at least one further light-emitting marker bound to a cell upon excitation by the second excitation wavelength λ2Ex.

In other embodiments, each light source may be associated with one or more detectors.

A first emission bandpass filter may be disposed in a light path between the flow channel and the first photodetector. The emission bandpass filter may have a transmission maximum in the range of 450-470 nm. It will be appreciated that a greater proportion of light having a peak emission wavelength falling within the transmission maximum of the bandpass filter will reach the first photodetector if the FWHM of this light is narrower.

The one or more further light-emitting markers contain a light-emitting material which is different from the light-emitting material of the first light-emitting marker and having a different peak wavelength.

For simplicity, FIG. 1 illustrates a flow cytometer having only two light sources and only two photodetectors however it will be understood that in other embodiments the flow cytometer may have more than two light sources and/or more than two photodetectors.

In some embodiments, a single light source may be configured to excite a single light-emitting marker of a plurality of light-emitting markers present in a sample being analysed or may be configured to excite a plurality of different light-emitting markers. It will therefore be appreciated that the flow cytometer may include only one light source.

In some embodiments, a sample to be analysed contains a plurality of light-emitting markers including a first light-emitting marker as described herein. Preferably, the first light-emitting marker has a full width at half maximum (FWHM) of less than 50 nm. Preferably, the first light-emitting marker has a peak emission wavelength which is separated by at least 50 nm from the peak of the light-emitting materials of the one or more further light-emitting markers.

Signals received by the forward scatter detector, side scatter detector and photodetectors may be transmitted by wired or wireless transmission to a signal processor (not shown).

### EXAMPLES

Measurements

Unless stated otherwise, emission spectra of light-emitting markers as described herein are as measured in methanol, using a Hamamatsu C₉₉₂₀₋₀₂ instrument having a set up wavelength 300 nm-950 nm; light source 150 W xenon light and bandwidth 10 nm or less (FWHM). Initially the system was calibrated with red (395 nm), green (375 nm) and blue (335 nm) glass standards. Two 5 ml long necked cuvettes (one filled with reference solvent i.e. water) and one filled with a sample of 1 mg/ml diluted 1 in 100 for a dissolved light-emitting marker or 1 mg/ml diluted ˜1 in 10 with water for a particulate light-emitting marker. The final concentration of the sample was altered to obtain a transmission data in the range 0.25-0.35. An average of 3 measurements for each sample is recorded.

Unless stated otherwise, absorption spectra of light-emitting materials as described herein are measured in water using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution. A baseline run with water in front and back 5 ml matched cuvettes (600 to 250 nm) following which the back cuvette reference remained as water and the front cuvette was changed to a sample of 1 mg/ml diluted 1 in 100 for a dissolved light-emitting marker or 1 mg/ml diluted ˜1 in 10 with water for a particulate light-emitting marker.

Unless stated otherwise, HOMO and LUMO levels and band gaps of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels of a conjugated polymer as described herein by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

For measurement of a polymer film, ferrocene is added directly to the existing cell at the end of the experiment for calculation purposes where the potentials are determined for the oxidation and reduction of ferrocene versus Ag/AgCl using cyclic voltammetry (CV).

LUMO=4.8-E ferrocene(peak to peak average)−E reduction of sample(peak maximum).

HOMO=4.8-E ferrocene(peak to peak average)+E oxidation of sample(peak maximum).

A typical SWV experiment runs at 15 Hz frequency; 25 mV amplitude and 0.004 V increment steps. Results are calculated from 3 freshly spun film samples for both the HOMO and LUMO data in the case of a polymer film, or from an average of 3 consecutive measurements of both HOMO and LUMO sweeps in the case of a solution.

All experiments are run under an Argon gas purge.

### Monomer Example 1

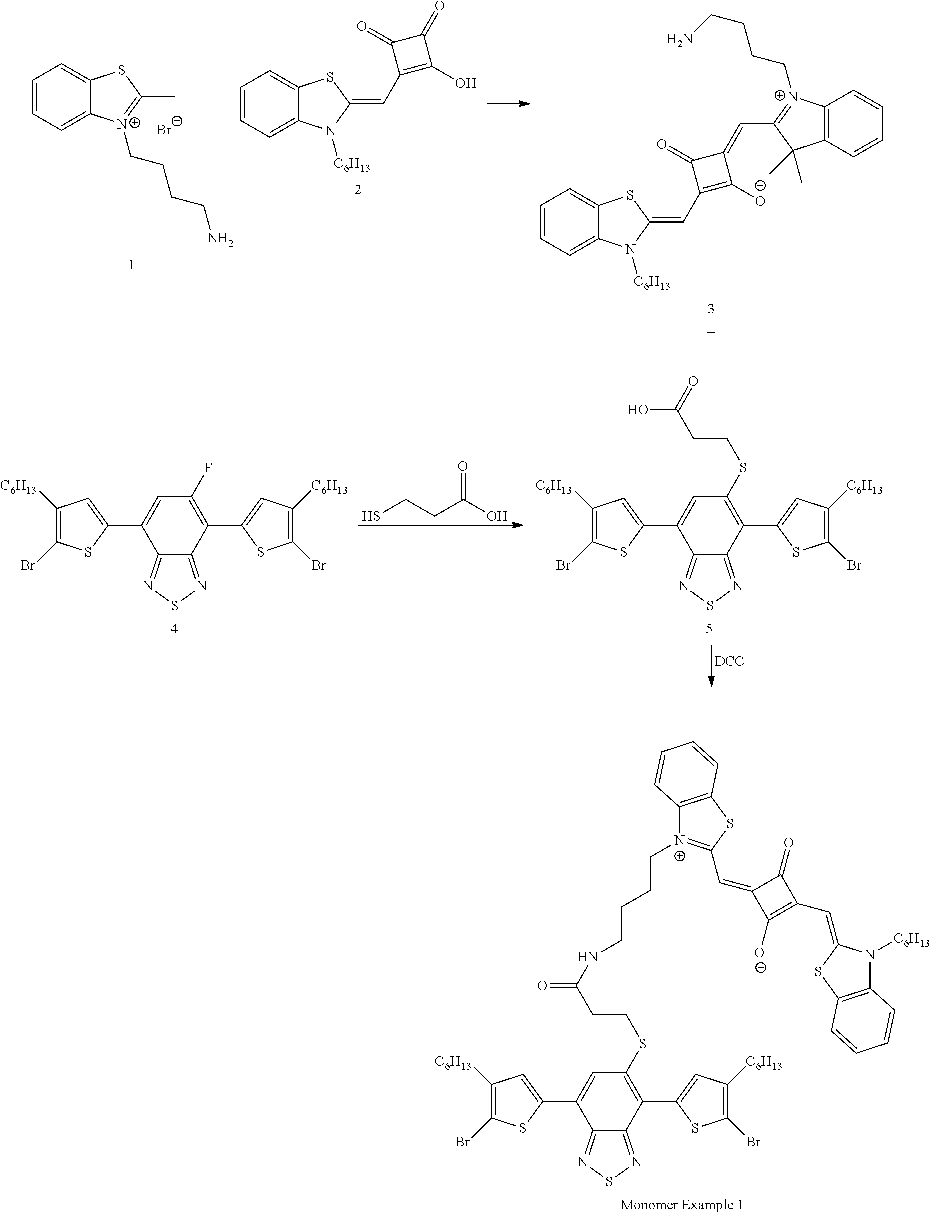

Intermediate 1 can be synthesised as outlined in CN108070275 or as per J. Phys Chem A, 2015, 119(52), 13025-13037 which describes an analogous compound.

Intermediate 2 can be synthesised as outlined in Langmuir, 2010, 26(16), 13486-13492 or by an analogous method to that laid out in Chem, Eur, J., 2013, 19(1), 218-232

Intermediate 3 can be synthesised by an analogous manner to that laid out in Chem, Eur, J., 2013, 19(1), 218-232.

Intermediate 4 can be synthesised as outlined in Polymer Chemistry, 2014, 5(22), 6551-6557.

Synthesis of 5

10 g (15.5 mmol) of intermediate 1 was dissolved in DMF (100 mL). Potassium carbonate (10.7 g, 77.5 mmol) was added and the reaction mixture cooled in an ice bath. 3-mercaptopropionic acid (8.1 mL, 93 mmol) was added dropwise and the reaction was heated to 50° C. and stirred for 3 h. After cooling the reaction mixture was poured into ice and stirred for 0.5 h. The precipitated solid was isolated by filtration. The crude material was recrystallized from toluene/acetonitrile and again from ethyl acetate/hexanes. The material was dried to yield 5.9 g, 52% yield and showed an HPLC purity of >99%.

Monomer Example 1 can be synthesized by a N,N′-dicyclohexane carbodiimide (DCC)-based amide formation reaction.

### Monomer Example 2

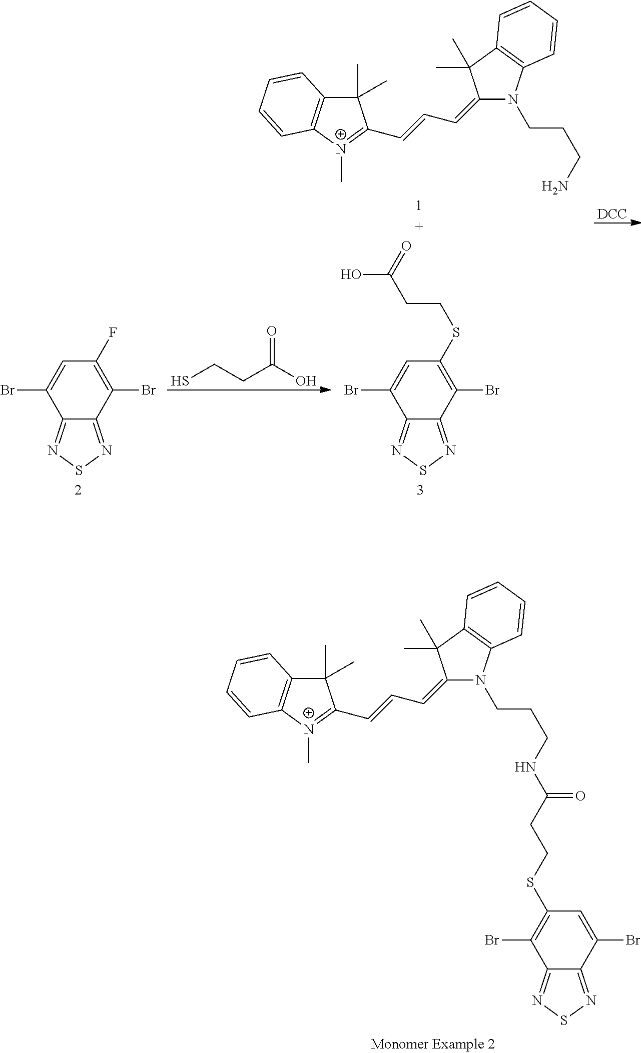

Intermediate 1 can be synthesized as outlined in U.S. Pat. No. 6,225,050

Intermediate 3 can be prepared as outlined in Polymer Chemistry, 2014, 5(22), 6551-6557

Monomer 2 can be synthesized by a N,N′-dicyclohexane carbodiimide (DCC)-based amide formation reaction

Synthetic Route to Monomer 3

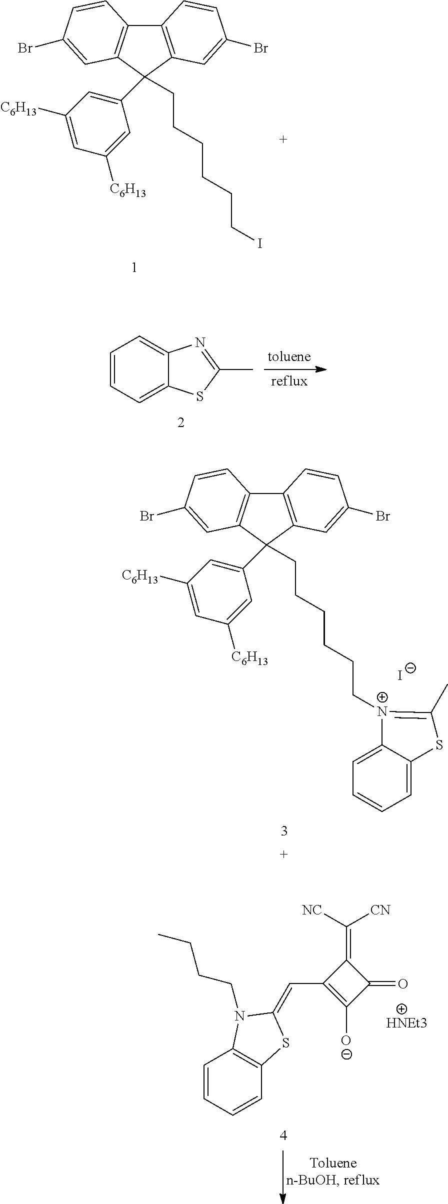

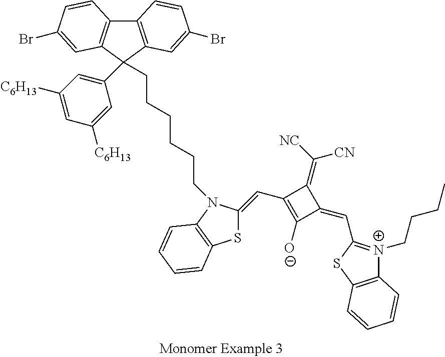

Intermediate 1 was synthesised as outlined in WO2012104628.

Intermediate 4 was synthesised as outlined in Angew. Chem. Int Ed, 2009, 48(46), 8776-8779.

Synthesis of Intermediate 3

Methylbenzothiazole (intermediate 2, 18.7 g, 125 mmol) was added to intermediate 1 (15 g, 19 mmol) in toluene with stirring. A clear solution was obtained at room temperature. The reaction mixture was heated to 120° C. for 16 hours. 100 mL diethyl ether was added to the viscous reaction mixture with stirring which produced a white precipitate which was filtered and washed with more diethyl ether. NMR was clean and corresponding to the desired the structure. LC-MS shows the M+ ion corresponding to intermediate 3.˜10 g (55% yield) intermediate 3 was obtained.

### Synthesis of Monomer Example 3

Intermediate 3 (2.9 g, 6.5 mmol) and Intermediate 3 (9.0 g, 9.7 mmol) were dissolved in degassed n-butanol (290 mL) and toluene (290 mL) in a reaction flask fitted with a Dean-Stark trap. The reaction mixture was further degassed for 0.5 h before being heated to 60° C. for 1 h and then 135° C. for 40 h. After cooling, the solvents were removed and the crude product was dissolved in DCM (200 mL) and precipitated into Methanol (2 L). The solid was filtered and purified by column chromatography on silica eluting with DCM. The product-containing fractions were combined and concentrated. The resulting solid was recrystallized from ethyl acetate/heptane to give 1 g of product as a white solid. HPLC showed 99.6% purity.

### Polymer Examples

Polymer Example 1 and Comparative Polymer 1 were prepared using Monomers A-D in which Monomers A and B are host monomers; Monomer C is an intermediate monomer; and Monomer D is an emissive monomer.

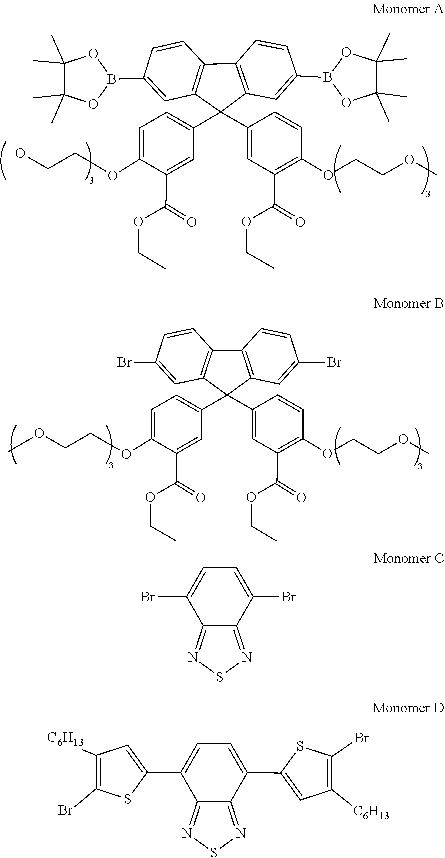

The formulations of Polymer Example 1 and Comparative Polymer 1 are shown below in Table 1.

Polymer Example 1 was synthesised and hydrolysed as described in U.S. Pat. No. 9,536,633 with the following modification: 10.6% Monomer A, 5% monomer C and 3% monomer D were incorporated into the reaction vessel for the first 2 hours of the reaction. After this time, the reaction was cooled <50° C. and the remaining 39.4% Monomer A and 42% Monomer B were added and the reaction reheated to reflux and completed as per the process described in U.S. Pat. No. 9,536,633. In this way, fluorene-benzothiadiazole and fluorene-bis(thienyl)benzothiadiazole directly linked to one another are formed within a first block and a chain of fluorene units is formed as a second block.

Comparative Polymer 1 was synthesised and hydrolysed as described out in U.S. Pat. No. 9,536,633. Comparative Polymer 1 uses the same loadings of monomers as Polymer 1 but all the monomers were added into the reaction vessel at the start of the reaction. This results in the fluorene-benzothiadiazole and fluorene-bis(thienyl)benzothiadiazole units randomly spread throughout the polymer chains.

| | Monomer A | Monomer B | Monomer C | Monomer D |
|---|---|---|---|---|
| | (block 1/ | (block 1/ | (block 1/ | (block 1/ |
| Polymer | block 2) | block 2) | block 2) | block 2) |
| Polymer | 50 | 42 | 5 | 3 |
| Example 1 | (10.6/39.4) | (0/42) | (5/0) | (3/0) |
| Comparative | 50 | 42 | 5 | 3 |
| Polymer 1 | (50/0) | (42/0) | (5/0) | (3/0) |

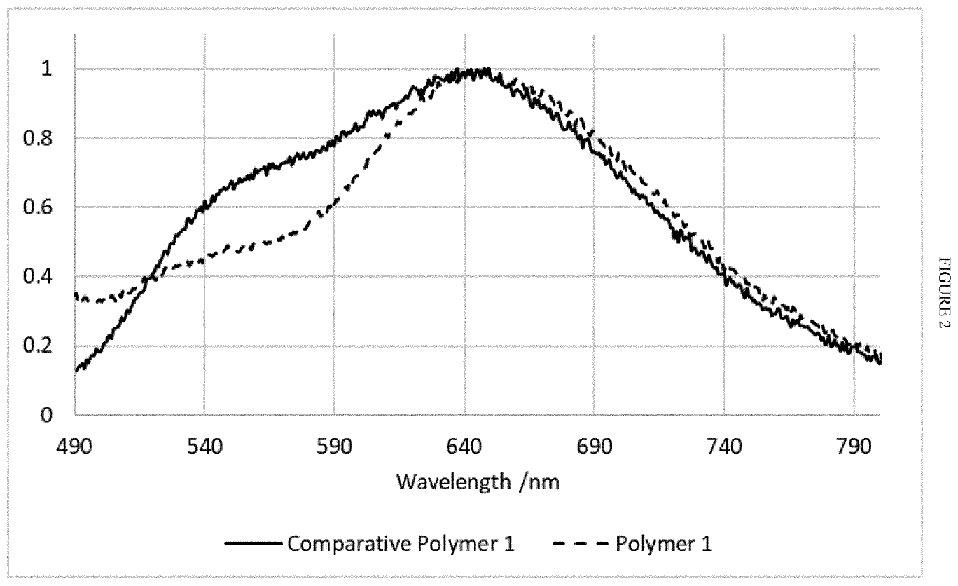

FIG. 2 shows emission spectra of Polymer Example 1 and Comparative Polymer 1.

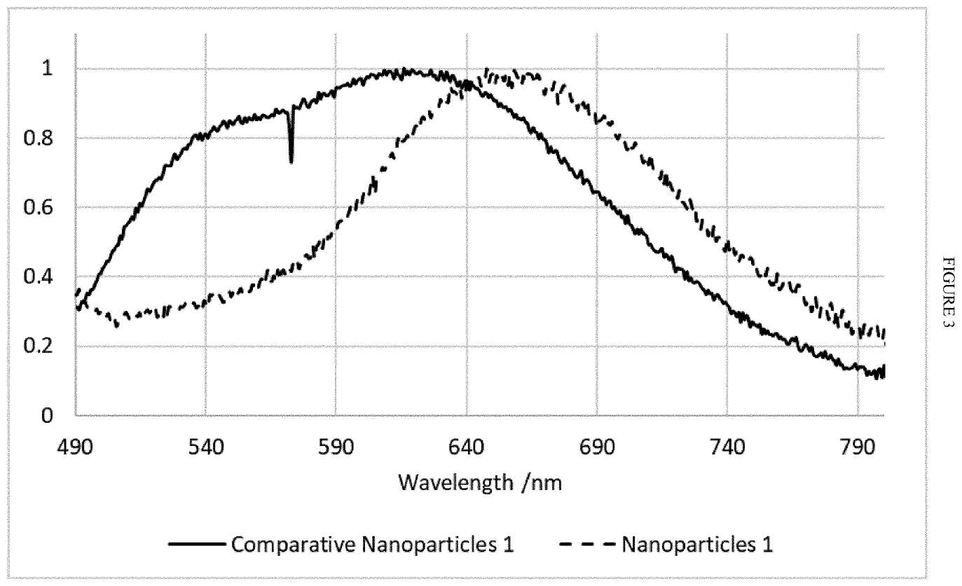

FIG. 3 shows spectra of particles containing Polymer Example 1 and Comparative Polymer 1.

As can be seen from FIGS. 2 and 3, the emission peak at ˜540 nm corresponding to emission from the intermediate unit is greatly reduced in Polymer 1 and Nanoparticles 1 compared to Comparative Polymer 1 and Comparative Nanoparticles 1 leaving mostly emission at ˜650 nm which corresponds emission from the emitter unit. Thus it is shown that arranging the intermediate units and emitter units are in close proximity in the polymer chain leads to better energy transfer to the emitter unit.

1. A conjugated light-emitting polymer comprising a host repeat unit and an intermediate repeat unit in a backbone of the conjugated light emitting polymer and an emissive unit wherein:
the host repeat unit has a wider band gap than the intermediate unit;
the intermediate repeat unit has a wider band gap than the emissive unit; and
the emissive unit is either:
an emissive repeat unit of a block copolymer wherein the block copolymer comprises a first block comprising the intermediate repeat unit and the emissive repeat unit and a second block comprising the host repeat unit;
a substituent of a proportion of the host repeat units and at least some of the host repeat units substituted with an emissive unit are arranged directly adjacent to an intermediate repeat unit;
a substituent of a proportion of the intermediate repeat units; or
a repeat unit in the backbone of the conjugated polymer and is arranged directly adjacent to the intermediate repeat unit.

2. The conjugated light-emitting polymer according to claim 1 wherein the emissive unit is a substituent of a proportion of the intermediate repeat units.

3. The conjugated light-emitting polymer according to claim 1 wherein the emissive unit is a substituent of a proportion of the host repeat units and at least some of the host repeat units substituted with an emissive unit are arranged directly adjacent to an intermediate repeat.

4. The conjugated light-emitting polymer according to claim 1 wherein the emissive unit is a repeat unit in the backbone of the polymer.

5. The conjugated light-emitting polymer according to claim 4 wherein the polymer is the block copolymer.

6. The conjugated light-emitting polymer according to claim 5 wherein the first block further comprises some of the host repeat units.

7. The conjugated light-emitting polymer according to claim 5 wherein the second block consists of the host repeat units.

8. The conjugated light-emitting polymer according to claim 1 wherein the host repeat unit is selected from a repeat unit of formula (I); a repeat unit of formula (II); and an arylene repeat unit which is unsubstituted or substituted with one or more substituents: 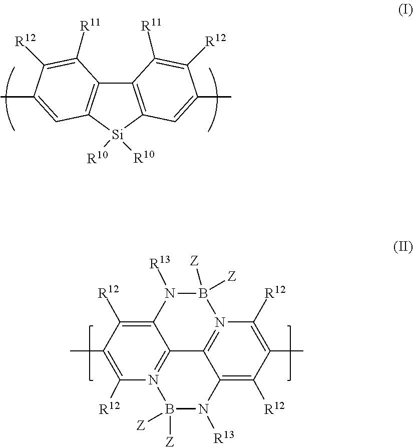
wherein R¹⁰ in each occurrence is independently a substituent; R¹¹ in each occurrence is independently H or a substituent and two R¹¹ groups may be linked to form a ring; R¹² independently in each occurrence is H or a substituent; R¹³ independently in each occurrence is a C₁₋₂₀ hydrocarbyl group; and Z in each occurrence is independently a substituent.

9. (canceled)

10. The conjugated light-emitting polymer according to claim 1 wherein the intermediate repeat unit comprises one or more heteroarylene units in the polymer backbone and wherein each of the one or more heteroarylene units is independently unsubstituted or substituted with one or more substituents.

11. The conjugated light-emitting polymer according to claim 10 wherein the intermediate repeat unit is selected from repeat units of formulae (IX)-(XII): 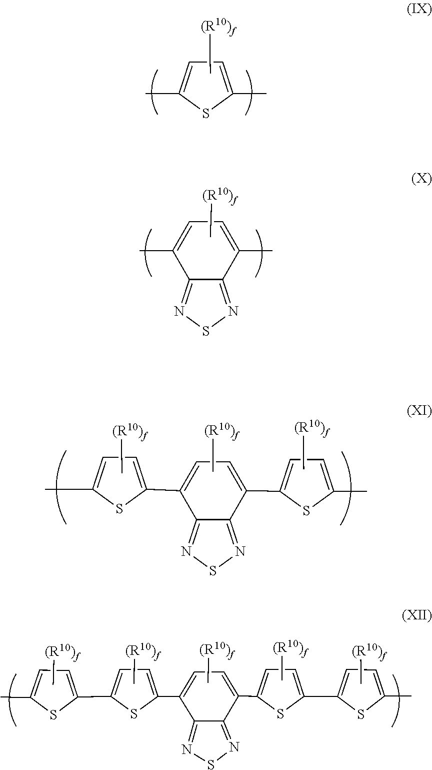
wherein R¹³ in each occurrence is independently a substituent, and f in each occurrence is independently 0, 1 or 2.

12. The conjugated light-emitting polymer according to claim 1 wherein the emissive unit has formula (XXX) or (XXXI): 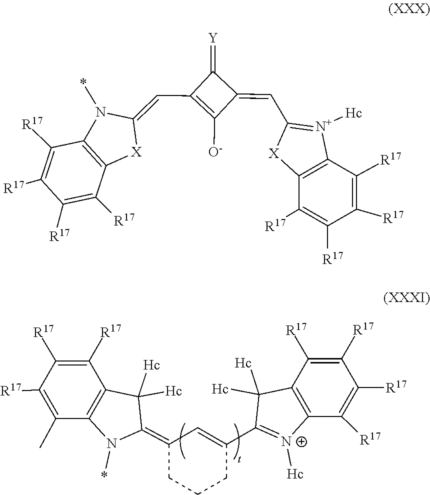
wherein Hc in each occurrence is independently a C₁₋₂₀ hydrocarbyl group and X is O, S or CR¹⁵₂ wherein R¹⁵ in each occurrence is independently H or a C₁₋₂₀ hydrocarbyl group; Y is O, S or C(CN)₂; t is 1, 2 or 3; R¹⁷ in each occurrence is H or a substituent and R¹⁷ groups linked to adjacent carbon atoms may be linked to form an aromatic or non-aromatic ring; and * represents a bond to a host repeat unit or an intermediate repeat unit or a divalent linking group L between the emissive unit and the host repeat unit or intermediate repeat unit.

13. The conjugated light-emitting polymer according to claim 1 wherein the host repeat units comprise more than 50 mol % of the repeat units of the polymer.

14-16. (canceled)

17. A light-emitting particle comprising the conjugated light-emitting polymer according to claim 1.

18. The light-emitting particle according to claim 17 wherein the particle comprises a matrix material.

19. The light-emitting particle according to claim 18 wherein the matrix material is silica.

20. A light-emitting marker comprising the conjugated light-emitting polymer according to claim 1 and a binding group comprising a biomolecule.

21. A precursor of the light-emitting marker according to claim 20 comprising a functional group capable of binding to the biomolecule.

22. (canceled)

23. A method of forming the light-emitting marker according to claim 20 comprising binding the biomolecule to a functional group of the precursor.

24. A formulation comprising the light-emitting marker according to claim 20 or a precursor thereof comprising a functional group capable of binding to a biomolecule wherein the light-emitting marker or precursor thereof is dissolved or dispersed in one or more solvents.

25. A method of identifying a target analyte in a sample, the method comprising irradiating the sample to which has been added a light-emitting marker according claim 20 configured to bind to the target analyte; and detecting emission from the light-emitting marker.

26. (canceled)

27. (canceled)
